(19) United States
(12) Patent Application Publication
(10) Pub. No.: US 2024/0268800 A1
(43) Pub. Date: Aug. 15, 2024

(54) REAL-TIME SAMPLING DEVICE

(71) Applicant: GYRUS ACMI, INC. D/B/A/ OLYMPUS SURGICAL TECHNOLOGIES AMERICA, Westborough, MA (US)

(72) Inventors: David A. Herrin, Seattle, WA (US); Eric Hadford, Maltby, WA (US); Dirk Tenne, Seattle, WA (US)

(21) Appl. No.: 18/410,979

(22) Filed: Jan. 11, 2024

Related U.S. Application Data
(60) Provisional application No. 63/444,495, filed on Feb. 9, 2023.

(51) Int. Cl. A61B 10/02

(52) U.S. Cl. CPC A61B 10/0233; A61B 1/2676; A61B 2010/0208; A61B 2010/045

(57) ABSTRACT

A sampling device insertable into a bronchoscope to obtain samples of target nodules in lungs of a patient, the system can include a sampling needle, a needle inlet guide tube, and a needle actuator. The sampling needle can extend between a base and a tip. The needle inlet guide tube can be removably couplable to the bronchoscope and can define a working lumen. The needle actuator can be configured to receive the base of the sampling needle to extend the sampling needle through the working lumen. The needle actuator can be engageable with the needle inlet guide tube to move the sampling needle through the sampling device. The sampling device can include operating states. In each of the operating states, the needle actuator can move relative to the needle inlet guide tube to move the tip of the sampling needle with respect to the sampling device.

### CLAIM OF PRIORITY

This patent application claims the benefit of priority, under 35 U.S.C. Section 119(e), to David Herrin U.S. Patent Application Ser. No. 63/444,495, entitled “REAL-TIME SAMPLING DEVICE,” filed on Feb. 9, 2023 (Attorney Docket No. 5409.824PRV), each of which is hereby incorporated by reference herein in its entirety.

### TECHNICAL FIELD

The present disclosure relates to a real-time sampling device. More specifically, the present disclosure relates to a real-time sampling device for collecting samples from within a body.

### BACKGROUND

Inserting and manipulating thin, elongated instruments within living bodies or other objects allows for ever-improving types of analysis, diagnosis, and treatment of those bodies or objects with minimally invasive techniques. By way of examples, noninvasive biopsies, endoscopic imaging, and catheterization treatments can enable evaluation and treatment of numerous internal lesions without invasive surgery.

Correspondingly, elongated instruments can also be used to collect samples from within a body in a relatively noninvasive matter. For example, when a biopsy from a lung is needed to determine whether a detected lesion is cancerous, instead of cutting into the chest of the patient to procure a sample, an insertion device such as a bronchoscope can be used to guide one or more elongated instruments to a location near the lesion to procure a sample. However, merely conveying the elongated instruments to the location near the lesion can present only a part of what is needed to sample the lesion itself.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 is a side view of a sampling device, according to at least one example of the present disclosure.

FIG. 2 is a cutaway view of a port in an actuator of a sampling device, according to at least one example of the present disclosure.

FIG. 3 is a side view of a needle actuator and an end cap of a sampling device, according to at least one example of the present disclosure.

FIG. 4 is a cutaway view of a needle actuator and an end cap of a sampling device, according to at least one example of the present disclosure.

FIG. 5 is a cutaway view of a needle actuator and an end cap of a sampling device, according to at least one example of the present disclosure.

FIG. 6 is a side view of a needle actuator and an end cap of a sampling device, according to at least one example of the present disclosure.

FIG. 7 is a side view of a cutaway portion of the sampling device showing a needle inlet guide tube and a needle actuator in a detached state, according to at least one example of the present disclosure.

FIG. 8 is a side view of a cutaway portion of the sampling device showing a needle inlet guide tube and a needle actuator in a locked state, according to at least one example of the present disclosure.

FIG. 9 is a side view of a cutaway portion of the sampling device showing a needle inlet guide tube and a needle actuator in an armed state, according to at least one example of the present disclosure.

FIG. 10 is a side view of a cutaway portion of the sampling device showing a needle inlet guide tube and a needle actuator in a standard sampling state, according to at least one example of the present disclosure.

FIG. 11 is a perspective view of a cutaway portion of the sampling device showing a needle inlet guide tube and a needle actuator in a standard sampling state, according to at least one example of the present disclosure.

FIG. 12 is a perspective view of a cutaway portion of the sampling device showing a needle inlet guide tube and a needle actuator with a second button assembly compressed as the sampling device moves into an extended sampling state, according to at least one example of the present disclosure.

FIG. 13 is a perspective view of a cutaway portion of the sampling device showing a needle inlet guide tube and a needle actuator in an extended sampling state, according to at least one example of the present disclosure.

FIG. 14 is a perspective view of a portion of the sampling device showing a locking mechanism adjacent to a second button assembly of a needle actuator, according to at least one example of the present disclosure.

FIG. 15 is a perspective view of a portion of the sampling device showing a locking mechanism on a second button assembly of a needle actuator, according to at least one example of the present disclosure.

FIG. 16 is a perspective view of a portion of the sampling device showing a locking mechanism on a second button assembly of a needle actuator within a portion of a housing, according to at least one example of the present disclosure.

FIG. 17 illustrates a flow diagram of an example of a method for real-time sampling, according to an example of the present disclosure.

FIG. 18A illustrates an example of a distal portion of an elongated instrument, according to at least one example of the present disclosure.

FIG. 18B illustrates an example of a distal portion of an elongated instrument, according to at least one example of the present disclosure.

FIG. 19A illustrates an example of a sample needle within an elongated instrument when a sampling device is in a locked state, according to at least one example of the present disclosure.

FIG. 19B illustrates an example of a sample needle within an elongated instrument when a sampling device is in a loaded state, according to at least one example of the present disclosure.

FIG. 19C illustrates an example of a sample needle within an elongated instrument when a sampling device is in a standard sampling state, according to at least one example of the present disclosure.

FIG. 19D illustrates an example of a sample needle within an elongated instrument when a sampling device is in a extended sampling state, according to at least one example of the present disclosure.

### DETAILED DESCRIPTION

It will be appreciated that various examples of the sampling device described herein can aid in the process of deploying and controlling an elongated instrument. The elongated instrument can include a flexible lumened catheter or a flexible lumened catheter that encompasses sampling needle. The flexible lumened catheter can be inserted into a body to target a tissue to be sampled. An imaging probe can be used to locate the tissue or a portion of the tissue of interest to be sampled. The imagine probe may be an ultrasound transducer that is integrated into the distal tip of the sampling device, or that is passed through a working channel of the sampling device and out of a port on the distal tip. The sampling needle can be extended beyond the flexible lumened catheter into the patient's tissue to procure a sample of that tissue.

The sampling device can be coupled with an insertion device, such as an endoscope or a bronchoscope, that can include an insertion conduit insertable into a body via an orifice or other opening (e.g., via a mouth into bronchial passageways). The sampling device can be coupled directly with the insertion device or coupled to a replaceable valve. The insertion device can enable the insertion conduit to be inserted into the body and directed to a desired location within the body. The insertion conduit can be configured to receive an elongated instrument that can be extendable through the insertion conduit. The elongated instrument can be insertable into the body via the insertion device to procure a tissue sample at the desired location within the body. The elongated instrument can be operably coupled with a control system that enables an operator to manipulate the elongated instrument to procure the tissue sample. An illustrative real-time sampling device including multiple operating states is described below.

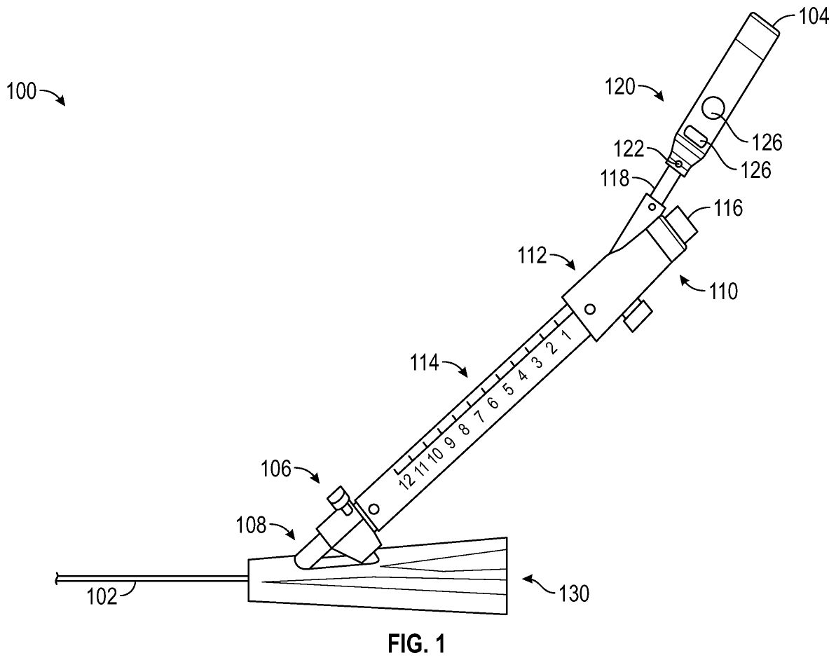

FIG. 1 is a side view of a sampling device 100, according to at least one example of the present disclosure. The sampling device 100 can operate in conjunction with an insertion device 130 (only a portion is shown in FIG. 1), such as an endoscope or a bronchoscope. As previously described, the insertion device 130 can include an insertion conduit that is insertable into a body via an orifice or other opening. In examples, the insertion device 130 can receive the elongated instrument 102, and the elongated instrument 102 can be extended through the insertion conduit to the desired location. For example, the elongated instrument 102 may be inserted through a working channel of a bronchoscope and extended from a port on the distal end of the bronchoscope. In this way, the elongated instrument 102 may be extended further into a bronchial structure than the bronchoscope due to the elongated instrument 102 having a smaller outer diameter than the bronchoscope. The elongated instrument 102 can be a sampling probe that can include an imaging probe (which may be integrated into a distal tip of the elongated instrument) and a sampling needle within a flexible lumened catheter. The elongated instrument 102 can be insertable via the insertion device 130 to procure a tissue sample at a desired location within a body. The elongated instrument 102 also can include a stylet that can be removably insertable into or through the needle, as further described below.

The sampling device 100 herein described can be coupled to the insertion device 130 using a coupling 106 at a distal end 108 of the sampling device 100. The elongated instrument 102, which can be manipulated by the sampling device 100, can extend through the coupling 106 and can be inserted into the insertion conduit of the insertion device 130. The elongated instrument 102 can be secured to an actuator 112 that is movably coupled to a housing 114. The actuator 112 can be moved along the housing 114 between a proximal end 110 and the distal end 108 of the sampling device 100 (which corresponds with proximal and distal ends of the housing 114) to extend and retract the elongated instrument 102 relative to the insertion device 130. Movement of the actuator 112 along the housing 114 in distal and proximal directions may cause the elongated instrument 102 to be extended distally from a port at the distal end of the insertion device 130 or retracted into the port, respectively. Anti-buckling devices can be received within the housing 114 to provide lateral support to the elongated instrument 102 as the actuator 112 moves the elongated instrument 102 through the housing 114.

In examples, the flexible lumened catheter of the elongated instrument 102 can be secured to the actuator 112 while the needle can be received into the flexible lumened catheter via the actuator 112. In some embodiments, a proximal port 116 can be configured to receive and secure an imaging probe such as, for example, a radial Endobronchial Ultrasound (EBUS) probe configured to generate real-time ultrasound images of tissue surrounding the distal end of the elongated instrument 102. A needle inlet guide tube 118 can be configured to receive and engage a needle actuator 120 to which the sampling needle can be secured. The needle inlet guide tube 118 and the needle actuator 120 can be movably coupled at an orientation interface 122. The orientation interface 122 can be configured to maintain an orientation of the needle actuator 120 relative to the needle inlet guide tube 118 to control an orientation of the sampling needle, as further described below. The needle actuator 120 can removably receive an end cap 124 that can be coupled with the stylet and used to releasably secure the stylet within the sampling needle. Depending on the location of target tissue within the patient's anatomy, the stylet may be used to prevent the sampling needle from collecting non-targeted tissue. For example, in a scenario in which the operator is targeting tissue several millimeters or centimeters beyond an airway wall, the operator may leave the stylet fully inserted in the sampling needle while the sampling needle is advance through non-target tissue. Then, when the operator sees on the real-time images generated by the imaging probe that the needle have reached or nearly reached the target tissue, the stylet may be withdrawn to permit target tissue to enter the sampling needle core. The needle actuator also can include a release mechanism 126 that an operator can positively engage to permit advancing the sampling needle into a sampling position, as also further described below.

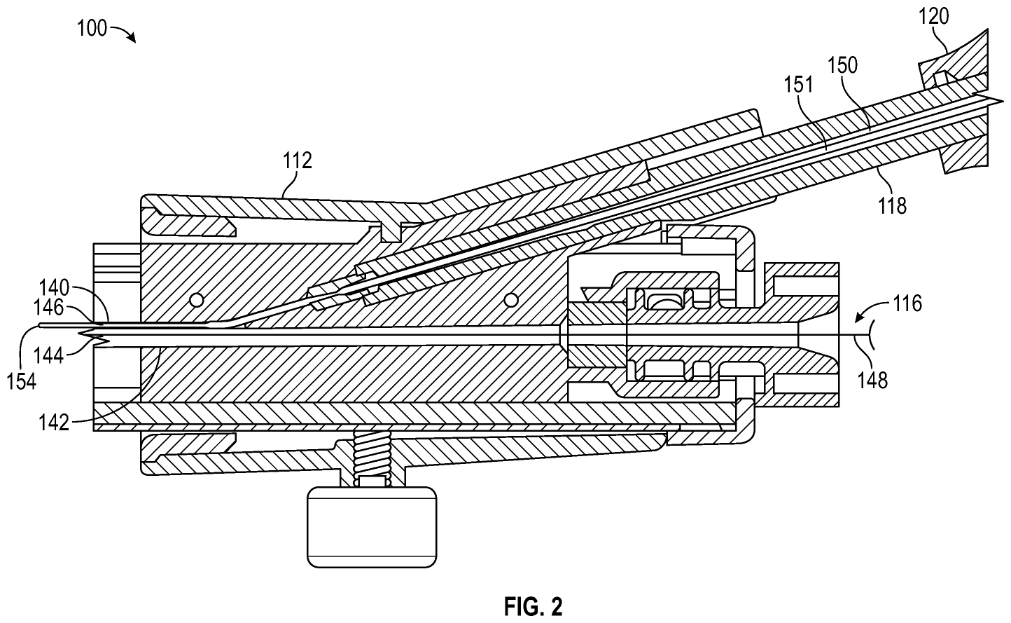

FIG. 2 is a cutaway view of the port 116 in the actuator 112 of the sampling device 100, according to at least one example of the present disclosure. The sampling device 100 can include an imaging probe 148. The proximal port 116 of the actuator 112 can be configured to receive the imaging probe 148 and direct the imaging probe 148 into a first lumen 144 of a flexible lumened catheter 140. The flexible lumened catheter 140 can include a proximal end 142 that can be coupled to the actuator 112. In examples, the flexible lumened catheter 140 can define a second lumen 146 configured to receive a sampling needle 150. The second lumen flexible lumened catheter 140 can extend into the first lumen 144 and can be configured to keep the sampling needle 150 away from the imaging probe 148 In other examples, the flexible lumened catheter 140 can define only a single lumen configured to receive the sampling needle 150 and a distal end of the elongated instrument 102 may include an imaging element (e.g., linear ultrasound transducer) integrated into the distal tip thereof adjacent to a ramp (of a side exit port) that is configured to directed the sampling needle 150 into a field of view of the imaging element. The sampling needle 150, as further described below, can be coupled with and controlled by the needle actuator 120.

The sampling needle 150 can extend between a base 152 (FIG. 4) and a tip 154. The sampling needle 150 can also include a lumen 151. The lumen 151 can be used to extract a sample from the patient. The needle actuator 120 can be slidably mounted on the needle inlet guide tube 118 (which can be also further described below). In examples, the sampling needle 150 can extend from the needle actuator 120 through the needle inlet guide tube 118 and into the second lumen 146 of the flexible lumened catheter 140, through which the sampling needle 150 can be extended into a body to collect a sample. The needle inlet guide tube 118 can also be joined with the actuator 112. Accordingly, when the needle inlet guide tube 118 and the imaging probe 148 are secured to the actuator 112, movement of the actuator 112 along the housing 114 can advance the elongated instrument 102 as well as the imaging probe 148 and sampling needle 150 contained therein.

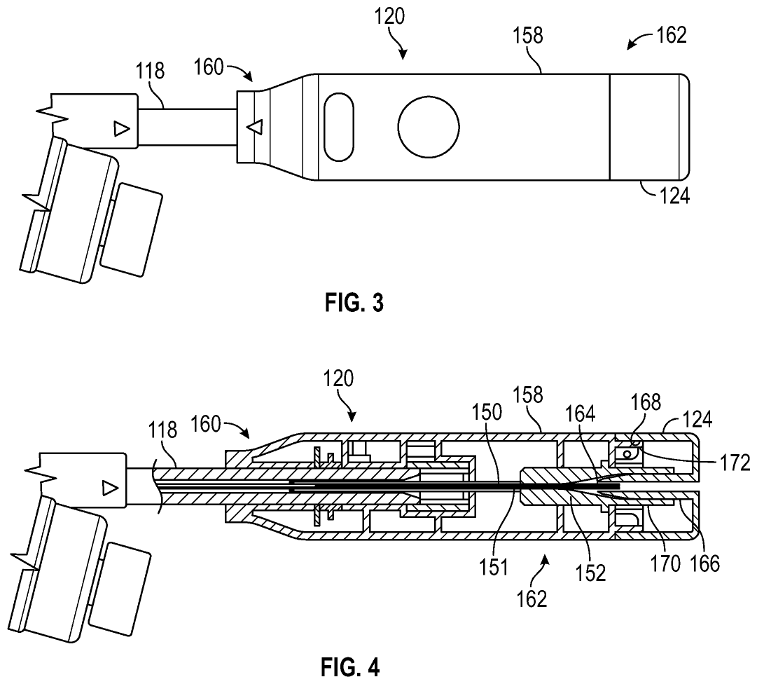

FIG. 3 is a side view of the needle actuator 120 and the end cap 124 of a sampling device 100, according to at least one example of the present disclosure. The sampling needle 150 can be secured and controlled (e.g., longitudinally advanced or retracted) by the needle actuator 120. The needle actuator 120 can include a housing 158. The housing 158 can have a distal end 160 that can engage the needle inlet guide tube 118. The needle actuator 120 can be movable along the needle inlet guide tube 118 to enable an operator to pierce or otherwise agitate a tissue with a distal end of the sampling needle 150 (FIG. 2) to retrieve a tissue sample. In examples, the end cap 124 can be removably securable to a proximal end 162 of the housing 158 of the needle actuator 120 to secure a stylet, as further described below.

FIG. 4 is a cutaway view of the needle actuator 120 and the end cap 124 of the sampling device 100, according to at least one example of the present disclosure. In examples, a stylet 164 can be movably received within the sampling needle 150, e.g., at the base 152, and can be fixably secured to the end cap 124. The stylet 164 can serve a range of functions including, by way of illustration and not limitation: sealing an end of the sampling needle 150 until the sampling needle 150 can be in position to collect a sample; adding stiffness to the sampling needle 150 to facilitate insertion into tissue; guiding or directing an end of the sampling needle 150; or other functions. Once the sampling needle 150 has been prepared to receive a tissue sample and includes the lumen 151, it can be desirable to withdraw the stylet 164 from the lumen 151 so that the tissue sample can be drawn into the lumen 151 of the sampling needle 150. To facilitate retrieval of the tissue sample, it can be desirable to apply a vacuum source such as a syringe or pump to a proximal end of the sampling needle 150 (via a proximal port, described below) once the stylet 164 can be withdrawn from the lumen 151 of the sampling needle 150.

In an example, an operator can move the needle actuator 120 along the needle inlet guide tube 118 to penetrate or agitate tissue at a distal end of the sampling needle 150. While the needle actuator 120 can be moved, the end cap 124 can cover the proximal end of the needle actuator 120. In addition, while the sampling needle 150 can be moved, it can be desirable to hold the stylet 164 in place so that agitation of the sampling needle 150 does not result in the stylet 164 inadvertently becoming dislodged and sliding out of the sampling needle 150 before it can be desirable to remove the stylet 164. The end cap 124 can help prevent the stylet 164 from dislodging.

In examples the stylet 164 can be secured in a stylet mount 166 of the end cap 124. The stylet 164 and at least a portion of the stylet mount 166 can both be receivable within a proximal port 170 at the proximal end 162 of the needle actuator 120. In examples, an inner surface 168 of the end cap 124 can be configured to engage an outer surface 172 of the proximal port 170 of the needle actuator 120 to secure the end cap 124 to the needle actuator 120 and, thus, hold the stylet 164 in place until withdrawal the stylet 164 is desired.

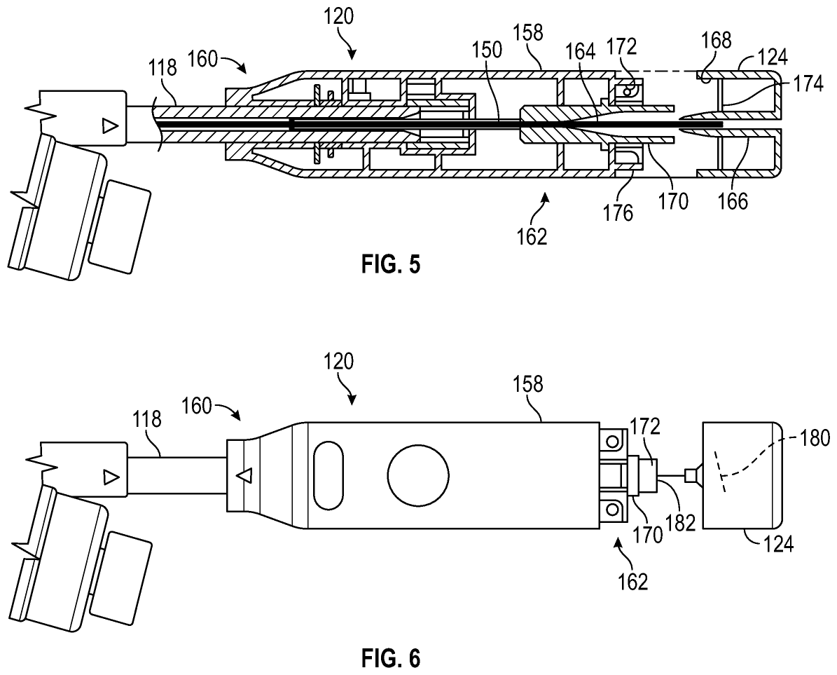

FIG. 5 is a cutaway view of the needle actuator 120 and the end cap 124 of the sampling device 100, according to at least one example of the present disclosure. In examples, the inner surface 168 of the end cap 124 can include a groove or other recess 174 that can be configured to engage a ridge or other projection 176 on the proximal port 170 of the needle actuator 120. The groove 174 can frictionally engage the ridge 176 such that a degree of force can be required of an operator to manually remove the end cap 124 when desired, where the degree of force is greater than an amount of force that can be applied to the stylet in the course of the sampling needle 150 being moved or agitated. In examples, the groove 174 or the ridge 176 can have curved or otherwise contoured cross-sections to facilitate engagement and disengagement of the groove 174 with the ridge 176 when the end cap 124 is installed and removed, respectively, from the proximal port 170.

FIG. 6 is a side view of the needle actuator 120 and the end cap 124 of the sampling device 100, according to at least one example of the present disclosure. In examples, the inner surface of the end cap 104 can also include one or more inner-facing threads 180 (represented by a dotted line) configured to engage one or more outer-facing threads 182 on the outer surface 172 of the proximal port 170. The threads 180 and 182 can enable the end cap 124 to be threadedly engaged with the proximal port 170 to alternately secure or remove the end cap 124 by rotating the end cap 124 relative to the housing 158 of the needle actuator 120. Once the end cap 124 is unsecured from the needle actuator 120, the stylet 164 can be withdrawn.

Although the examples of FIGS. 4-6 show the end cap 124 engaging the proximal port 170 to secure the end cap 124 to the needle actuator 120, it will be appreciated that the end cap 124 can engage other parts of the housing 158 of the needle actuator 120 to secure the stylet 164 in place during manipulation of the needle actuator 120.

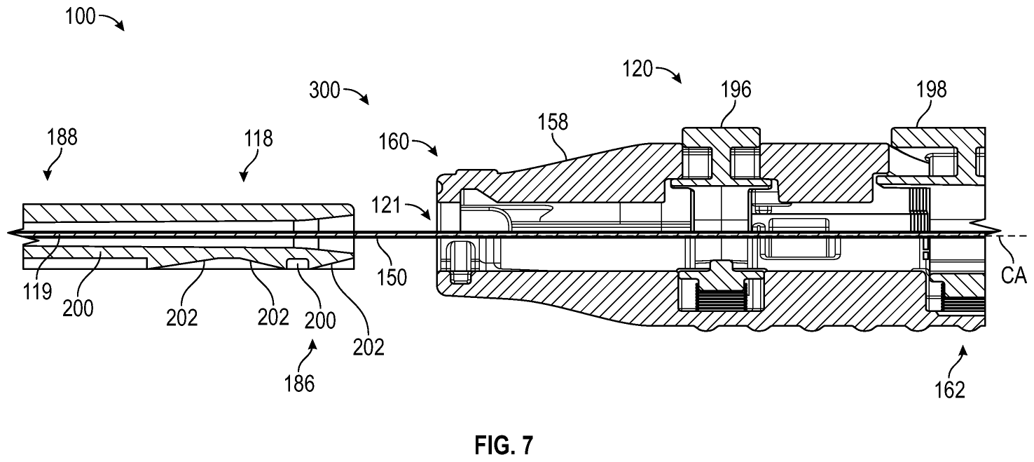

FIG. 7 is a side view of a cutaway portion of the sampling device 100 showing a needle inlet guide tube 118 and a needle actuator 120 in a detached state 300, according to at least one example of the present disclosure. As discussed above, the sampling device 100 can include a sampling needle 150 extending between a base 152 (FIG. 4) and a tip 154 (FIG. 2) and a needle actuator 120 configured to be coupled to the base 152 of the sampling needle 150. The sampling device 100 can also include a needle inlet guide tube 118 that can be configured to slidably convey the sampling needle 150 from a proximal end of the needle inlet guide tube 118 to a distal end of the needle inlet guide tube 118. The needle inlet guide tube 118 can define a working lumen 119 therein.

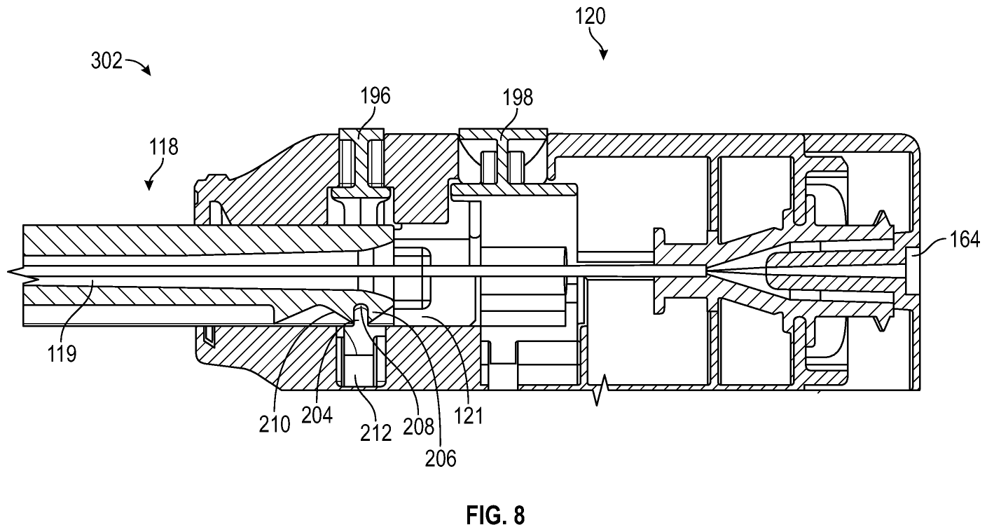

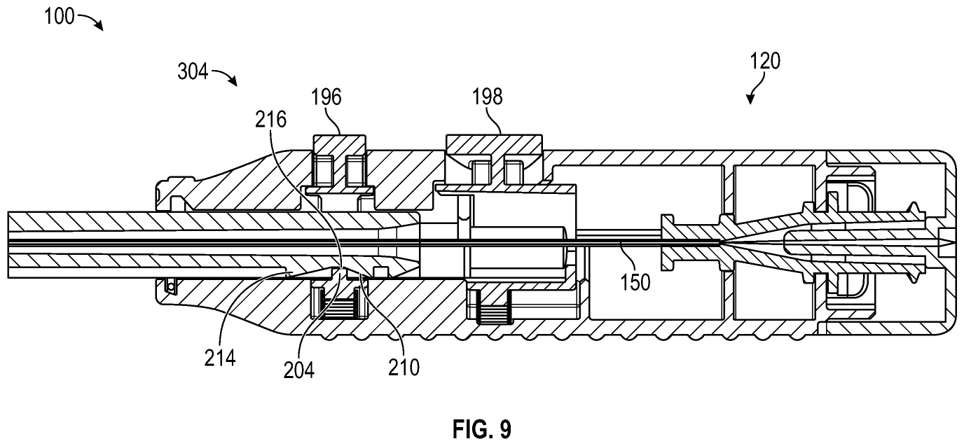

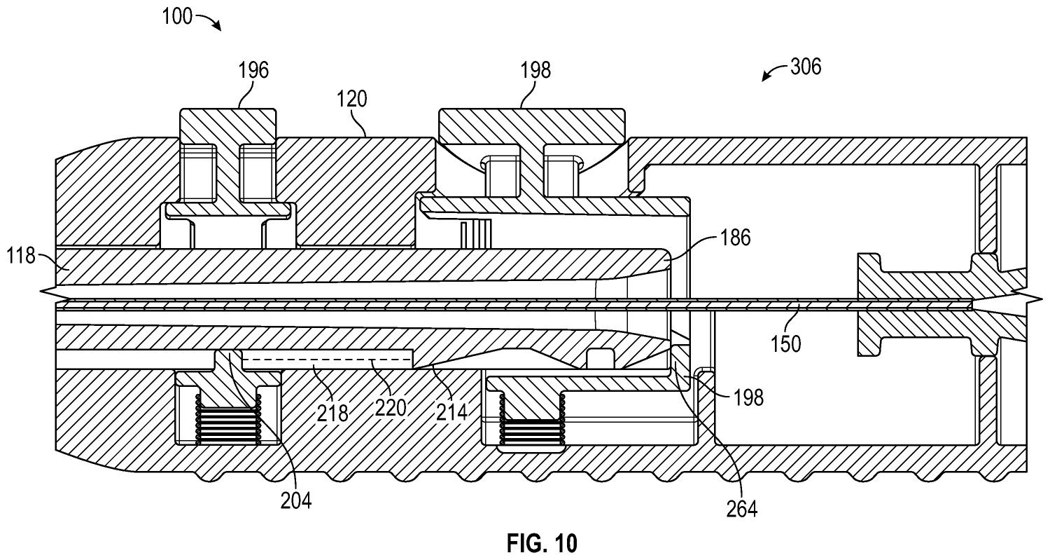

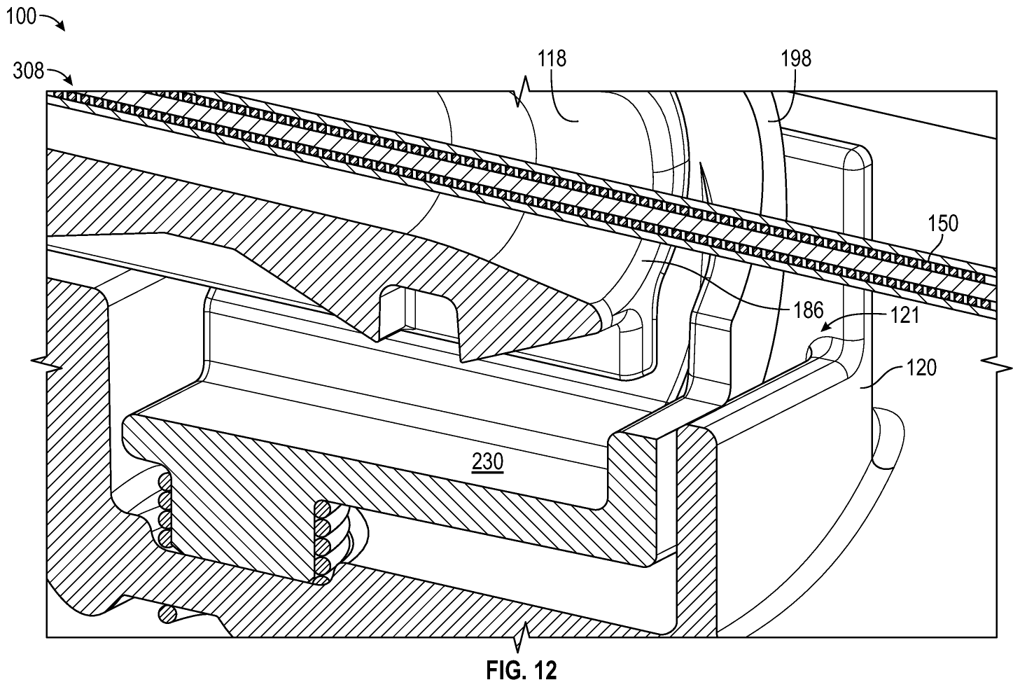

In an example, the needle inlet guide tube 118 can extend longitudinally between a third end portion 186 and a fourth end portion 188 such that the third end portion 186 can be insertable into a working lumen 121 of the needle actuator 120. The needle inlet guide tube 118 can be configured to engage with the needle actuator 120 to move the needle actuator 120 relative to the needle inlet guide tube 118 between different states. These different states may include a detached state (shown in FIG. 7), a locked state (shown in FIG. 8), an armed state (shown in FIG. 9), a standard sampling state (shown in FIG. 10), and an extended sampling state (shown in FIG. 12).

As shown in FIG. 7, the needle inlet guide tube 118 can be detached from the needle actuator 120. In the detached state 300, the sampling needle 150 can be inserted and advanced through the working lumen 119 to help guide the sampling needle 150 into the elongated instrument 102 (FIG. 1). For example, the needle inlet guide tube 118 can direct the sampling needle 150 into the second lumen 146 (FIG. 2) of the flexible lumened catheter 140 (FIG. 2).

The needle actuator 120 can include a housing 158 that can extend longitudinally along a central axis CA between the distal end 160 and the proximal end 162. The release mechanism 126 can include a first button assembly 196 and a second button assembly 198. The first button assembly 196 can extend at least partially outside of the housing such that the first button assembly 196 can be engageable from outside the housing 158. The second button assembly 198 can extend at least partially outside of the housing 158 such that the second button assembly 198 can be engageable from outside the housing 158.

In an example such as that shown in FIG. 7, in the detached state 300, the needle actuator 120 and the needle inlet guide tube 118 can be separated from one another such that the needle actuator 120 and the needle inlet guide tube 118 are not touching. For example, maintenance can be conducted on the sampling device 100 before the needle actuator 120 is attached to the needle inlet guide tube 118. For example, a new needle or a needle of a different size can be installed within the needle actuator 120. The detached state 300 can also enable cleaning and reprocessing of the sampling device 100, for example, to replace wear components and to update and sterilize any components of the sampling device 100 before the next operation.

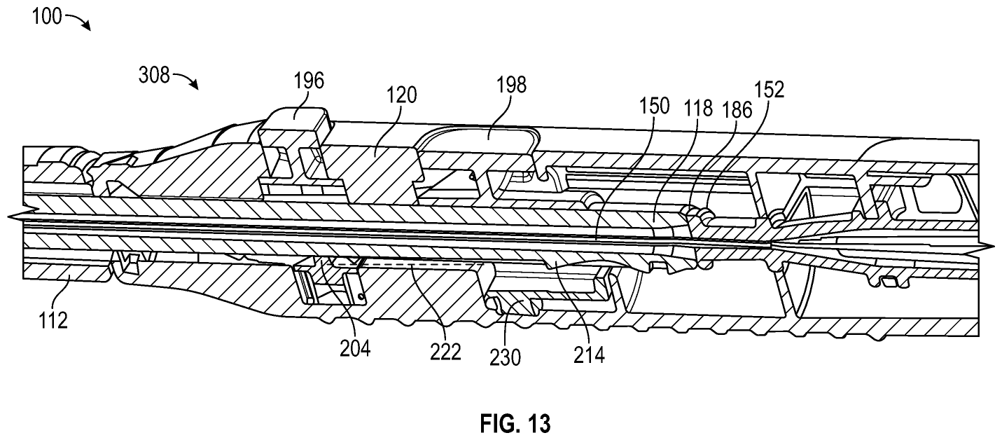

The sampling device 100 can operate between the detached state 300, a locked state 302 (an example of which is shown in FIG. 8), an armed state 304 (an example of which is shown in FIG. 9), a standard sampling state 306 (an example of which is shown in FIG. 10), and the extended sampling state 308 (an example of which is shown in FIG. 13). In reference to FIG. 7, the needle inlet guide tube 118 can include at least one groove 200 configured to engage with the needle actuator 120. The needle inlet guide tube 118 can also include at least one engagement ramp 202 configured to engage with the needle actuator 120 as the sampling device moves between the detached state 300, the locked state 302, the armed state 304, the standard sampling state 306, and the extended sampling state 308.

FIG. 8 is a side view of a cutaway portion of the sampling device 100 showing the needle inlet guide tube 118 and the needle actuator 120 in the locked state 302, according to at least one example of the present disclosure.

The first button assembly 196 can include a first engagement feature 204 that can be engageable with the needle inlet guide tube 118. In an example, a spring 212 can bias the first button assembly 196 such that the first engagement feature 204 at least partially extends into the working lumen 121 of the needle actuator 120. In an example, the needle inlet guide tube 118 can include a first engagement ramp 206 adjacent to the third end portion 186 of the needle inlet guide tube 118, a first groove 208 adjacent to the first engagement ramp 206, and a second engagement ramp 210 adjacent to the first groove 208.

The first engagement ramp 206 can extend radially outward from a central axis of the needle inlet guide tube 118 as the first engagement ramp 206 extends from the third end portion 186 toward the fourth end portion 188. The first groove 208 can extend radially inward from the first engagement ramp 206 and extend longitudinally toward the fourth end portion 188. A length of the first groove 208 can be configured to be complementary with one or more engagement features of the needle actuator 120 such that the one or more engagement features of the needle actuator 120 can catch within the first groove 208 to prevent translation of the needle actuator 120 relative to the needle inlet guide tube 118. The second engagement ramp 210 can extend radially inward toward a central axis of the needle inlet guide tube 118 as the second engagement ramp 210 extends from the first groove 208 toward the fourth end portion 188. In the example shown in FIG. 8, the first engagement ramp 206 and the second engagement ramp 210 are symmetric about the first groove 208. In another example, the first engagement ramp 206 and the second engagement ramp 210 can be asymmetrical about the first groove 208.

The first engagement feature 204 of the first button assembly 196 can include a protrusion, ridge, or any other feature that can engage with and catch into the engagement features of the needle inlet guide tube 118. The width of the first engagement feature 204 can be configured to be complementary with at least one groove of the needle inlet guide tube 118.

In an example, the first engagement feature 204 can engage with the first engagement ramp 206 or the second engagement ramp 210 and can catch in the first groove 208 to move the sampling device 100 into the locked state 302. In the locked state 302, the needle actuator 120 cannot move or translate, or can have very limited movement, relative to the needle inlet guide tube 118. When the sampling device 100 is in the locked state 302, the tip of the sampling needle 150 can be retracted five or more millimeters within the sampling device 100. For example, if the flexible lumened catheter 140 (FIG. 2) is within the elongated instrument 102 (FIG. 1), the sampling needle 150 can be retracted five or more millimeters within the elongated instrument 102 or the flexible lumened catheter 140. If the flexible lumened catheter 140 is extended from the elongated instrument 102, the sampling needle 150 can be retracted within the flexible lumened catheter 140 by five or more millimeters.

In an example, to toggle the sampling device 100 between the detached and locked states, the needle actuator 120 can be slid over the needle inlet guide tube 118 until the first button assembly 196 contacts the first engagement ramp 206. As the needle actuator 120 slides over the needle inlet guide tube 118 and the needle actuator 120 advances, the first engagement feature 204 can ride up the first engagement ramp 206 and compresses the spring 212 of the first button assembly 196. The compression of the spring 212 can generate resistance against the needle actuator 120 sliding over the needle inlet guide tube 118. As the first engagement feature 204 clears the first engagement ramp 206, the spring 212 can decompress to press the first engagement feature 204 into the first groove 208. As the first engagement feature 204 is within the first groove 208, the sampling device 100 is in the locked state 302, which can limit translation of the needle actuator 120 relative to the needle inlet guide tube 118.

In the locked state 302, the spring 212 can maintain the first engagement feature 204 in the first groove 208 such that the actuator 112 cannot translate with relation to the needle inlet guide tube 118. Thus, in the locked state 302, the sampling needle 150 is contained within the sampling device 100 and cannot advance outside the elongated instrument 102 or the flexible lumened catheter 140. The sampling device 100 can stay in the locked state 302, until the first button assembly 196 is engaged by an operator to remove the first engagement feature 204 from the first groove 208. Therefore, the locked state 302 can help prevent accidental extension of the tip of the sampling needle 150 outside of the elongated instrument 102 or the flexible lumened catheter 140 (FIG. 2). In this way, while the sampling device 100 is in the locked state, an operator may advance or retract the elongated instrument 102 from a working channel of a bronchoscope that is inserted into a patient's airway toward a target tissue, or otherwise manipulate the elongated instrument 102 within the patient, without inadvertently causing tissue trauma as a result of the sampling needle inadvertently extending from an exit port at the distal end of the elongated instrument 102.

FIG. 9 is a side view of a cutaway portion of the sampling device 100 showing the needle inlet guide tube 118 and the needle actuator 120 in the armed state 304, according to at least one example of the present disclosure.

In an example, the needle inlet guide tube 118 can include a third engagement ramp 214 and a trough feature 216 between the second engagement ramp 210 and the third engagement ramp 214. The trough feature 216 can extend longitudinally from the second engagement ramp 210 toward the fourth end portion 188. In examples, the trough feature 216 can maintain a consistent thickness of the needle inlet guide tube 118 such that the trough feature 216 does not extend radially inward or radially outward as the trough feature 216 extends longitudinally. Stated alternatively, the surface of the trough feature 216 may be parallel to the central axis (e.g., longitudinal axis) of the needle inlet guide tube 118. The third engagement ramp 214 can extend radially outward from the central axis of the needle inlet guide tube 118 as the third engagement ramp 214 extends from the trough feature 216 toward the fourth end portion 188.

A length of the third engagement ramp 214 can be altered to change a stroke length required to move the sampling device 100 from the armed state 304 and into the standard sampling state 306. For example, a larger stroke length can require more translation of the needle actuator 120 with respect to the needle inlet guide tube 118 to move the sampling device 100 from the armed state 304 and into the standard sampling state 306, and a smaller stroke length can require less translation of the needle actuator 120 with respect to the needle inlet guide tube 118 to put the sampling device 100 from the armed state 304 and into the standard sampling state 306. In another example, the third engagement ramp 214 can have a flat spot in a portion of the third engagement ramp 214 that is nearest the fourth end portion 188. The flat portion of the third engagement ramp 214 can help indicate when the sampling device 100 is moving between the armed state 304 and the standard sampling state 306.

In an example, the first button assembly 196 can be engaged to slide the needle actuator 120 relative to the needle inlet guide tube 118 such that the first engagement feature 204 can engage with the second engagement ramp 210 or the third engagement ramp 214. The needle actuator 120 can translate along the needle inlet guide tube 118 until the first engagement feature 204 catches in the trough feature 216, thereby biasing the first engagement feature 204 onto the trough feature 216 by virtue of the second engagement ramp 210 and the third engagement ramp 214 each leading to the trough feature 216.

Once the first engagement feature 204 catches in the trough feature 216 the sampling device 100 is in the armed state 304. In the armed state 304, no more buttons, e.g., the first button assembly 196, the second button assembly 198, or the like, need to be engaged to put the sampling device 100 into the standard sampling state 306. In an example, the tip of the sampling needle 150 can be retracted within the sampling device 100 about three to five millimeters when the sampling device 100 is in the armed state 304. For example, if the flexible lumened catheter 140 is within elongated instrument 102, the sampling needle 150 can be retracted within the flexible lumened catheter 140 (FIG. 2) or the elongated instrument 102 (FIG. 1) about three to five millimeters. If the flexible lumened catheter 140 is extended beyond the elongated instrument 102, the sampling needle 150 can be retracted within the flexible lumened catheter 140 about three to five millimeters.

In an example, from the locked state 302 the operator can engage the first button assembly 196 to move the first engagement feature 204 out of the first groove 208. While the first engagement feature 204 is out of the first groove 208, the operator can advance the needle actuator 120 by translating the needle actuator 120 with relation to the needle inlet guide tube 118. Once the first engagement feature 204 clears the most radially outward portion of the second engagement ramp 210 the sampling device 100 is in the armed state 304. In the armed state 304, the needle actuator 120 can translate with relation to the needle inlet guide tube 118 between the portion of the second engagement ramp 210 adjacent to the first groove 208 and the portion of the third engagement ramp 214 nearest the fourth end portion 188 (FIG. 7).

In the armed state 304, the operator can advance the needle actuator 120 relative to the needle inlet guide tube 118 such that the first engagement feature 204 rides up the third engagement ramp 214. As the first engagement feature 204 rides up the third engagement ramp 214, the spring 212 of the first engagement ramp 206 can be compressed. The compression of the spring 212 can increase a resistance to translation of the needle actuator 120 relative to the needle inlet guide tube 118 as the needle actuator 120 and the needle inlet guide tube 118 get closer to leaving the armed state 304 and entering the standard sampling state 306. Such a resistance to translation of the needle actuator 120 relative to the needle inlet guide tube 118 can provide feedback to an operator that they are leaving the armed state 304 and entering the standard sampling state 306 (FIG. 10), which can help reduce accidental movements from the armed state 304 to the standard sampling state 306.

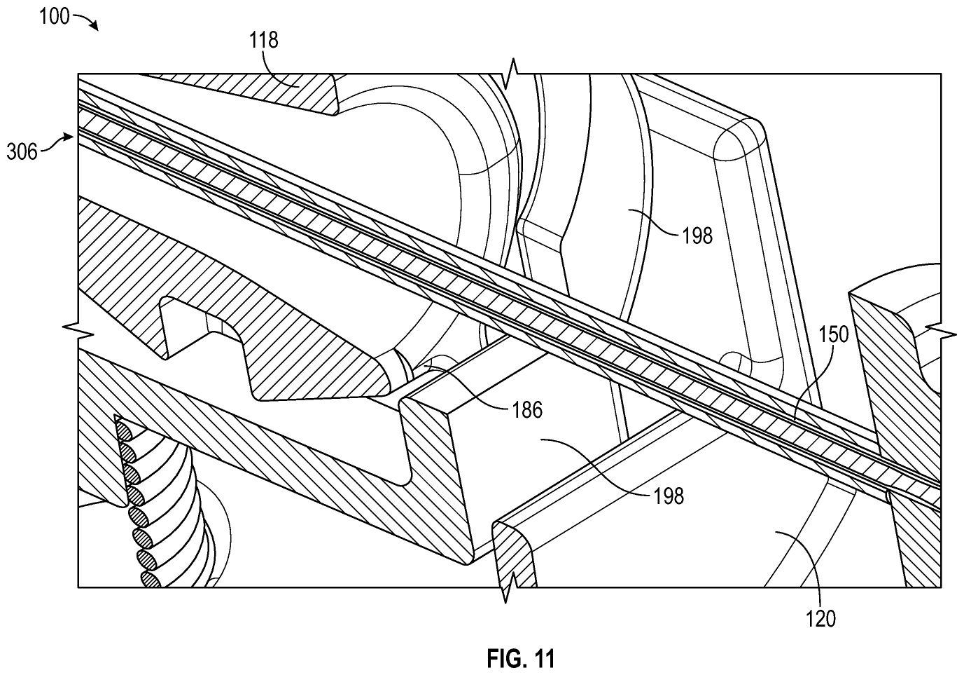

FIGS. 10 and 11 will be discussed together below. FIG. 10 is a side view of a cutaway portion of the sampling device 100 showing the needle inlet guide tube 118 and the needle actuator 120 in the standard sampling state 306, according to at least one example of the present disclosure. FIG. 11 is a perspective view of a cutaway portion of the sampling device 100 showing the needle inlet guide tube 118 and the needle actuator 120 in the standard sampling state 306, according to at least one example of the present disclosure.

The needle inlet guide tube 118 can include a standard sampling groove 218 adjacent to the third engagement ramp 214 and extending toward the fourth end portion 188. The standard sampling groove 218 can extend from the third engagement ramp 214 all the way to the fourth end portion 188 without extending radially inward or outward such that there is no change in resistance as the needle actuator 120 translates relative to the needle inlet guide tube 118. In an example, sliding the needle inlet guide tube 118 relative to the needle actuator 120 such that the first engagement feature 204 can engage the third engagement ramp 214 and the first engagement feature 204 can catch in the standard sampling groove 218, puts the sampling device 100 in the standard sampling state 306.

In the standard sampling state 306, the needle actuator 120 can translate back and forth, e.g., proximally and distally, with relation to the needle inlet guide tube 118. The back-and-forth translation of the needle actuator 120 relative to the needle inlet guide tube 118 can permit the operator to move the sampling needle 150 relative to a distal tip flexible lumened catheter 140 (FIG. 2). The movement of the tip, e.g., the tip 154 (FIG. 2) of the sampling needle 150 relative to a distal tip of the flexible lumened catheter 140 permits the sampling needle 150 to extend within the lungs or any other body part of a patient, beyond the travel limit of the elongated instrument 102 or the flexible lumened catheter 140, respectively. The increased extension of the sampling needle 150, as compared to the elongated instrument 102 or the flexible lumened catheter 140, can permit the sampling of a nodule of the lung of the patient that is beyond the reach of the elongated instrument 102 or the flexible lumened catheter 140. In yet another example, the sampling device 100 in the standard sampling state 306 can be used before the elongated instrument 102 or the flexible lumened catheter 140 has reached their maximum insertion point to decrease the amount the elongated instrument 102 or the flexible lumened catheter 140 needs to extend within the body of the patient, while still being able to take a sample of the tissue of the patient with the sampling needle 150.

In the armed state 304, the sampling device 100 can have limits to how far the sampling needle 150 can extend from the sampling device 100 in the standard sampling state 306. In one example, a first travel limit 220 (represented by a dashed line in FIG. 10) can be defined by the first engagement feature 204 engaging with the third engagement ramp 214 and the third end portion 186 of the needle inlet guide tube 118 engaging with the second button assembly 198. For example, as shown in FIG. 10, an internal protrusion 264 of the second button assembly 198 makes contact with the third end portion 186, which prevents the needle actuator 120 from moving any further distally than the end of the first travel limit 220. However, depressing the second button assembly 198, as described below and shown in FIGS. 12 and 13, provides clearance between the internal protrusion 264 and the third end portion 186 of the needle inlet guide tube 118—thereby enabling the needle actuator 120 to be further advanced into the extended sampling state. The first travel limit 220 can allow the tip of the sampling needle 150 to extend from the elongated instrument 102 or the flexible lumened catheter 140 between zero and ten millimeters when the sampling device 100 can be in the standard sampling state. The first travel limit 220 can be configured to reduce the likelihood of accidental puncture, e.g., pneumothorax, or the like. Additionally, because the airways of a patient are typically very small, the first travel limit 220 between 0 and 10 millimeters is typically sufficient to sample most nodules of the lungs. The first travel limit of 0 to 10 millimeters is provided for example purposes only, and may be adjusted depending on clinical application parameters.

In an example, the operator can slide the needle actuator 120 relative to the needle inlet guide tube 118 to put the sampling device 100 in the standard sampling state 306. The operator can slide the needle actuator 120 relative to the needle inlet guide tube 118 back and forth the extend and retract the sampling needle 150 from the flexible lumened catheter 140 (FIG. 2), or any other distal edge of the elongated instrument 102, to take a sample of the nodule of the lung or any other tissue of the patient. When the sampling needle 150 reaches the target and a sample is obtained, the operator can engage the first button assembly 196 and go directly into the detached state 300 (FIG. 7), in which the sampling needle 150 can be directly removed from the sampling device 100. This is because when the first button assembly 196 is fully depressed, the first engagement feature 204 is clear of each of the engagement ramps 206, 210, 214. The operator can then expel the sample onto a slide for pathology examination. After adequate samples have been captured, the sampling device 100 can be removed from the bronchoscope or any other scope used to carry the sampling device 100 within the patient.

FIGS. 12 and 13 will be discussed together below. FIG. 12 is a perspective view of a cutaway portion of the sampling device 100 showing the needle inlet guide tube 118 and the needle actuator 120 with the second button assembly 198 compressed as the sampling device 100 moves into the extended sampling state 308, according to at least one example of the present disclosure. FIG. 13 is a perspective view of a cutaway portion of the sampling device 100 showing the needle inlet guide tube 118 and the needle actuator 120 in the extended sampling state 308, according to at least one example of the present disclosure.

In an example, an operator can engage the second button assembly 198 such that a second base 230 of the second button assembly 198 can retract outside of the working lumen 121. After the second base 230 is retracted outside of the working lumen 121 the sampling device 100 is in the extended sampling state 308. As such, the operator can slide the needle actuator 120 relative to the needle inlet guide tube 118 such that the third end portion 186 of the needle inlet guide tube 118 can clear the second base 230 and the sampling device 100 can move from the standard sampling state 306 and into the extended sampling state 308.

In the extended sampling state 308, the needle actuator 120 can move back and forth, e.g., distally and proximally, relative to the needle inlet guide tube 118 a distance limited by a second travel limit 222. In an example, the second travel limit 222 can be defined by the first engagement feature 204 engaging with third engagement ramp 214 and the third end portion 186 engaging with the base 152 of the sampling needle 150. In another example, the second travel limit 222 can also be defined by the needle actuator 120 contacting the actuator 112. In an example, the second travel limit can allow the tip of the sampling needle 150 to extend from the flexible lumened catheter 140 (FIG. 2) between zero and twenty millimeters when the sampling device 100 is in the extended sampling state 308.

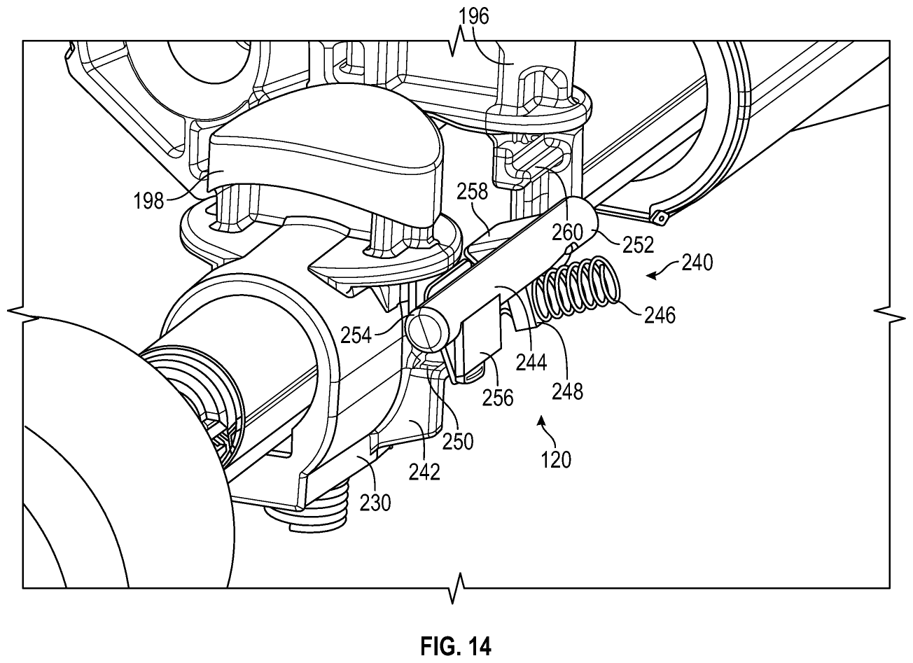

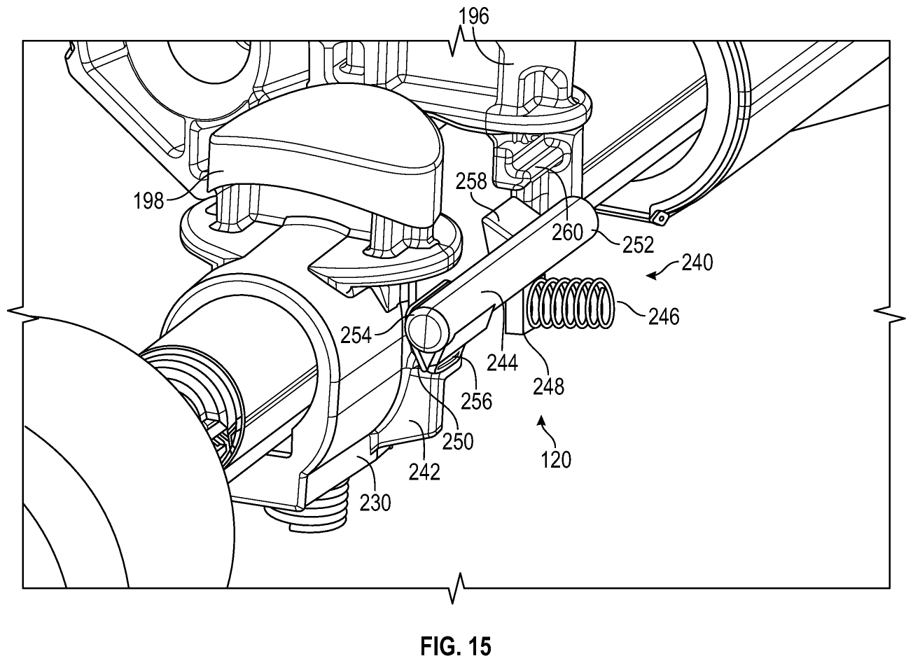

FIGS. 14-16 will be discussed together below. FIG. 14 is a perspective view of a portion of the sampling device 100 showing a locking mechanism 240 on the second button assembly 198 of the needle actuator 120, according to at least one example of the present disclosure. FIG. 15 is a perspective view of a portion of the sampling device 100 showing the locking mechanism 240 on the second button assembly 198 of the needle actuator 120, according to at least one example of the present disclosure. FIG. 16 is a perspective view of a portion of the sampling device 100 showing the locking mechanism 240 on the second button assembly 198 of the needle actuator 120, according to at least one example of the present disclosure.

The second button assembly 198 can include a lock stop 242 having a lock surface 250. The lock stop 242 can extend laterally outward from the second base 230. The lock surface 250 can be engageable to maintain the second button assembly 198 in an engaged position. The locking mechanism 240 of the second button assembly 198 can include a pivotable rod 244, a spring base 248, and a lock catch 256. The locking mechanism 240 can be cradled in the housing 158 (shown partially in phantom in FIG. 16) of the needle actuator 120 and configured to maintain the second button assembly 198 in the engaged position after the second button assembly 198 is engaged by an operator of the sampling device 100. The pivotable rod 244 can extend from a first end 252 to a second end 254. The pivotable rod 244 can be configured to rotate the locking mechanism 240 in a clockwise and counterclockwise direction.

The lock catch 256 can be generally triangular and can extend from the second end 254 of the pivotable rod 244. The lock catch 256 can be configured to engage with the lock stop 242 to prevent the pivotable rod 244 from rotating clockwise when the second button assembly 198 is not compressed and can be configured to engage with the lock surface 250 to keep the second button assembly 198 compressed once the second button assembly 198 is engaged by an operator.

The spring base 248 can extend from the first end 252 of the pivotable rod 244 in a direction that is circumferentially offset from the lock catch 256. The locking mechanism 240 can also include a locking spring 246 that can be attached to the spring base 248 and can be configured to extend between the spring base 248 and an interior wall of the housing 158. The locking spring 246 and the spring base 248 can be configured to rotate the pivotable rod 244 and the locking mechanism 240 in the clockwise direction when the second button assembly 198 is compressed. For example, when the second button assembly 198 is engaged by an operator and moves into the compressed position, the lock stop 242 can clear the lock catch 256. The locking spring 246 can rotate the pivotable rod 244 clockwise to position the lock catch 256 above the lock surface 250 such that the lock catch 256 engages the lock surface 250 when the operator stops engaging the second button assembly 198 (as shown in FIG. 15).

In an example, an unlock flag 258 can also extend from the first end 252 of the pivotable rod 244. The unlock flag 258 can be engageable to rotate the locking mechanism 240 in the counterclockwise direction. In an example, the first button assembly 196 can include an extended sampling state exit tab 260 that can extend longitudinally from the first button assembly 196 toward the distal end 160 of the housing 158. The extended sampling state exit tab 260 can be engageable with the unlock flag 258 of the locking mechanism 240 to rotate the locking mechanism 240 in the counterclockwise direction to disengage the lock catch 256 from the lock surface 250 of the lock stop 242.

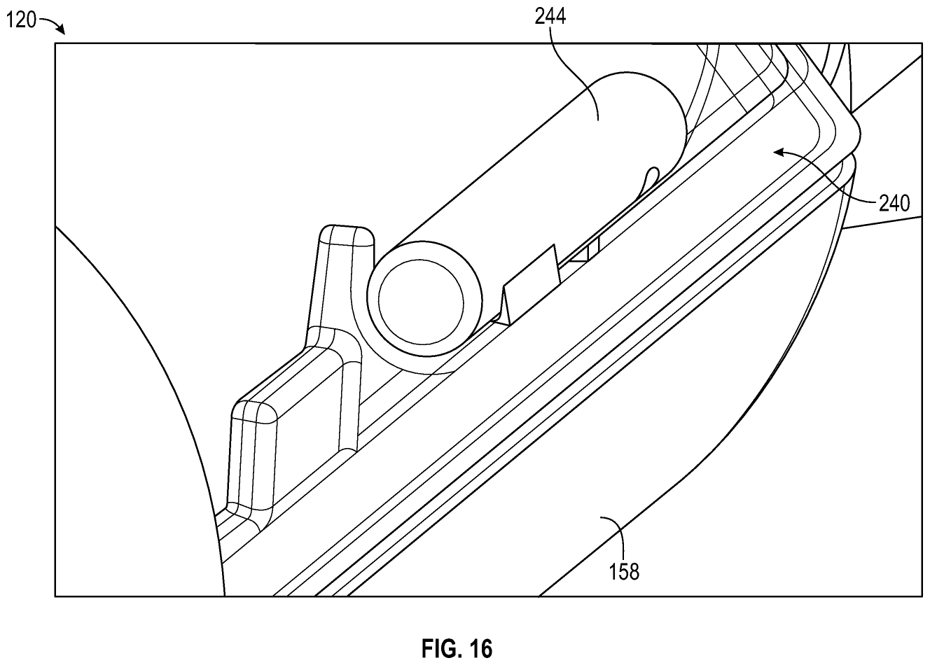

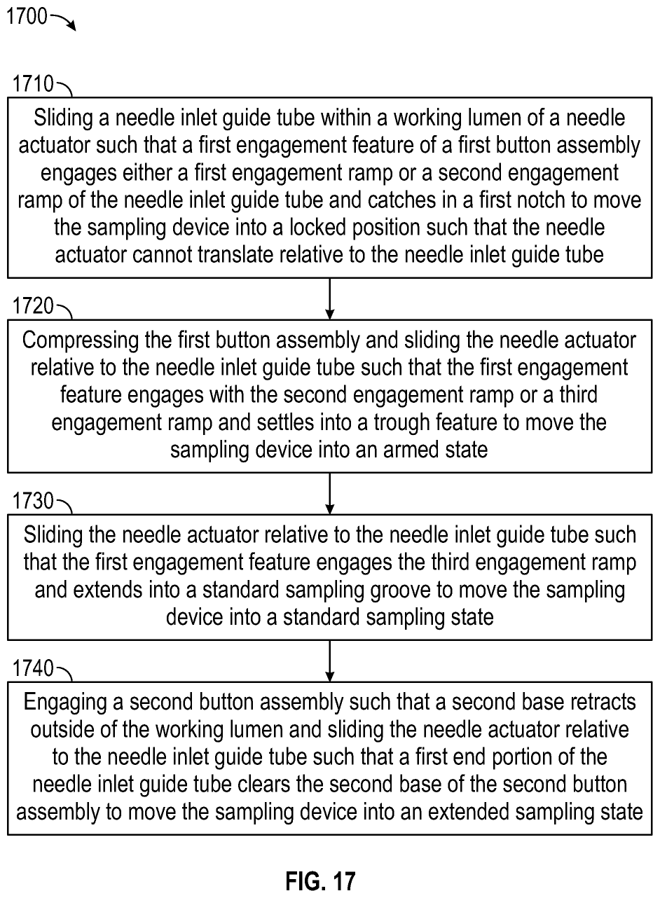

FIG. 17 illustrates a schematic view of the method 1700, in accordance with at least one example of this disclosure. The method 1700 can be a method of real-time sampling. The steps or operations of the method 1700 are illustrated in a particular order for convenience and clarity; many of the discussed operations can be performed in a different sequence or in parallel without materially impacting other operations. The method 1700 as discussed can include operations performed by multiple different actors, devices, or systems. It is understood that subsets of the operations discussed in the method 1700 can be attributable to a single actor, device, or system could be considered a separate standalone process or method.

In an example, at step 1710, the method 1700 can include sliding a needle inlet guide tube, e.g., the needle inlet guide tube 118 discussed herein, within a working lumen, e.g., the working lumen 121 discussed herein, of a needle actuator, e.g., the needle actuator 120 discussed herein, such that a first engagement feature, e.g., the first engagement feature 204 discussed herein, of a first button assembly, e.g., the first button assembly 196 discussed herein, engages either a first engagement ramp or a second engagement ramp, e.g., the at least one engagement ramp 202, the first engagement ramp 206, or the second engagement ramp 210 discussed herein, of the needle inlet guide tube and catches in a first notch, e.g., the at least one groove 200 or the first groove 208, to move the sampling device into a locked position such that the needle inlet guide tube cannot move relative to the needle actuator.

In an example, at step 1720, the method 1700 the method can include compressing the first button assembly and sliding the needle actuator relative to the needle inlet guide tube such that the first engagement feature engages with the second engagement ramp or a third engagement ramp and settles into a trough feature to move the sampling device into an armed state.

At step 1730, the method 1700 can include sliding the needle actuator relative to the needle inlet guide tube such that the first engagement feature engages the third engagement ramp and extends into a standard sampling groove to move the sampling device into a standard sampling state.

At step 1740, the method 1700 can include engaging a second button assembly such that a second base retracts outside of the working lumen and sliding the needle actuator relative to the needle inlet guide tube such that a first end portion of the needle inlet guide tube clears the second base of the second button assembly to move the sampling device into an extended sampling state. In an example, a standard travel limit can allow a tip of a sampling needle to extend from a housing of the needle actuator between zero to ten millimeters when the sampling device can be in the standard sampling state. In an example, an extended travel limit can allow a tip of the sampling needle to extend from the housing between zero to twenty millimeters when the sampling device can be in the extended sampling state.

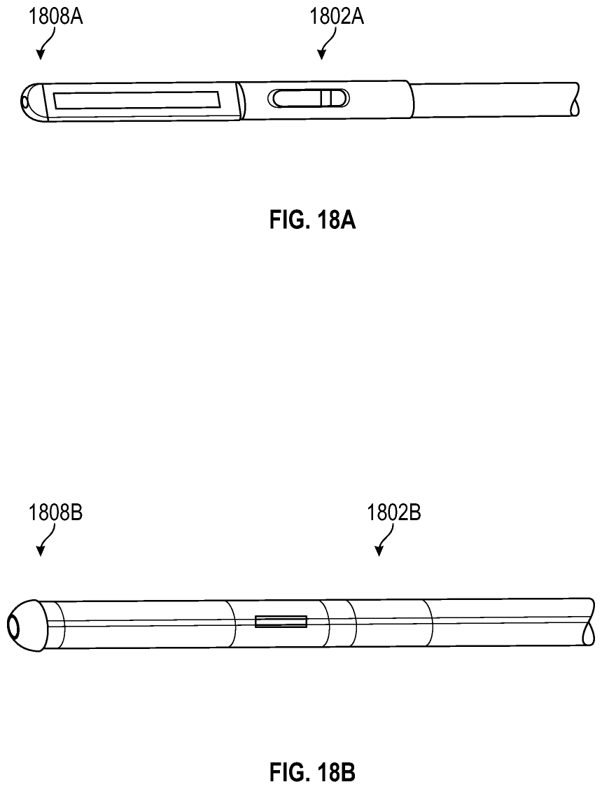

FIG. 18A illustrates an example of a distal portion 1808A of an elongated instrument 1802A, according to at least one example of the present disclosure. FIG. 18B illustrates an example of a distal portion 1808B of an elongated instrument 1802B, according to at least one example of the present disclosure. FIGS. 18A and 18B will be discussed together below.

As shown in FIG. 18A, the distal portion 1808A of the elongated instrument 1802A can include an imaging probe (e.g., imaging probe 148 (FIG. 2)). As shown in FIG. 18A, the imaging probe can be an ultrasonic transducer that is imbedded into the distal portion 1808A of the elongated instrument 1802A. As shown in FIG. 18B, the distal portion 1808B of the elongated instrument 1802B can include an aperture from which the imaging probe or transducer can be extended. Moroever, a portion of the distal portion 1808B can be windowed such that the image sensor can reside within the elongated instrument 1802B while radially transmitting an ultrasound signal. The distal portion 1808A of elongated instrument 1802A and the distal portion 1808B of the elongated instrument 1802B are shown for illustrative purposes, and are not intended to limit the disclosed systems to just these examples of the elongated instruments. For example, the systems described herein can be used with any elongated instrument installed into the patient that needs a controlled limit of extension of a medical instrument beyond the extension of the system within the body. For example, any other medical instrument (e.g., a scalpel, forceps, any other medical instrument, or any combination thereof) can be attached to the systems described herein to control the system between a detached state, a locked state, an armed state, a standard sampling state, and an extended sampling state. In examples, such a system can be inserted into a patient without the use of a bronchoscope or any other device, such that the elongated member of the system can be inserted directly into the patient, and the system can be used to control the extension of the medical instrument therefrom the elongated member.

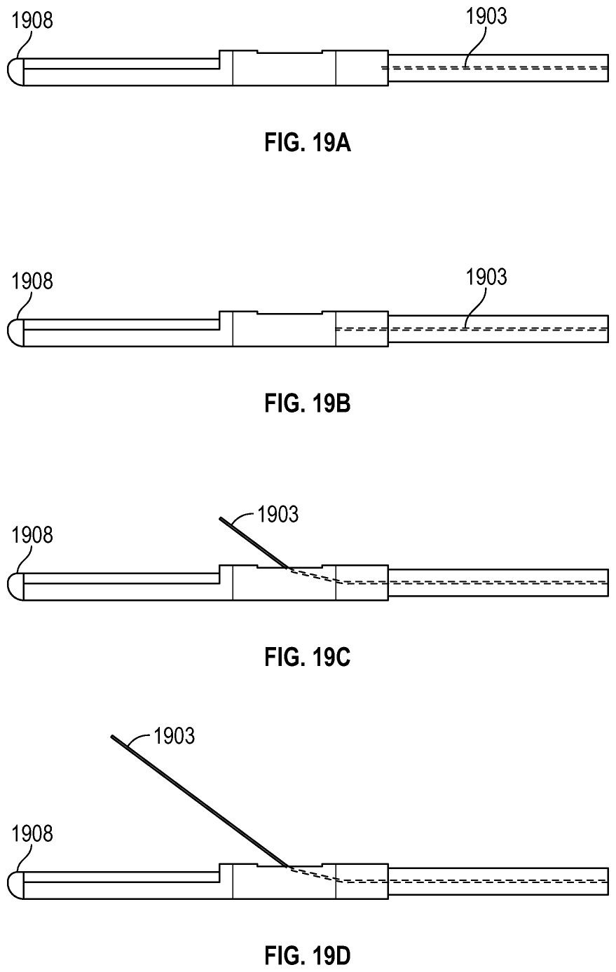

FIG. 19A illustrates an example of a sample needle within an elongated instrument when a sampling device is in a locked state, according to at least one example of the present disclosure. FIG. 19B illustrates an example of a sample needle within an elongated instrument when a sampling device is in a loaded state, according to at least one example of the present disclosure. FIG. 19C illustrates an example of a sample needle within an elongated instrument when a sampling device is in a standard sampling state, according to at least one example of the present disclosure. FIG. 19D illustrates an example of a sample needle within an elongated instrument when a sampling device is in a extended sampling state, according to at least one example of the present disclosure.

As discussed above, each state (e.g., the detached state 300, the locked state 302, the armed state 304, the standard sampling state 306, and the extended sampling state 308) can move the distal tip (e.g., the tip 154 (FIG. 2)) of the sampling needle or any other medical instrument 1903 with relation to a distal portion 1908 of the elongated instrument.

As shown in FIG. 19A, the instrument 1903 can be positioned such that the entirety of the medical instrument is within the elongated member when the system is in the locked state. As shown in 19B, the instrument 1903 can still be positioned within the medical instrument, but closer to a perimeter (either the distal end or a side) thereof the elongated member, when the system is in the loaded state.

As shown in FIG. 19C, the medical instrument 1903 can extend out of the distal portion 1908 of the elongated instrument. For example, as shown in FIG. 19C, the instrument 1903 can be guided out a side of the elongated instrument. In another example, the instrument 1903 can extend out of an aperture in the distal end of the elongated instrument when the system is in the standard sampling state.

As shown in FIG. 19D, the instrument 1903 can extend even further outside the elongated member, when the system is in the extended state. As shown in FIG. 19D, the medical instrument 1903 can extend out of the distal portion 1908 of the elongated instrument. For example, as shown in FIG. 19C, the instrument 1903 can be guided out a side of the elongated instrument. In another example, the instrument 1903 can extend out of an aperture in the distal end of the elongated instrument when the system is in the extended state. As shown in FIG. 19D, the instrument 1903 can extend about twice as far out of the elongated instrument when the sampling system is in the extended state as compared to the standard sampling state. In another example, the ratio of extension of the medical instrument can be altered with respect to the standard sampling state and the extended sampling state.

The following, non-limiting examples, detail certain aspects of the present subject matter to solve the challenges and provide the benefits discussed herein, among others.

Example 1 is a sampling device insertable into a bronchoscope to obtain samples of target nodules in lungs of a patient, the sampling device comprising: a sampling needle extending between a base and a tip; a needle inlet guide tube removably couplable to the bronchoscope and defining a working lumen; and a needle actuator configured to receive the base of the sampling needle to extend the sampling needle through the working lumen, the needle actuator engageable with the needle inlet guide tube to move the sampling needle through the sampling device; the sampling device including operating states, in each of the operating states the needle actuator moves relative to the needle inlet guide tube to move the tip of the sampling needle with respect to the sampling device.

In Example 2, the subject matter of Example 1 includes, wherein the operating states comprise: a detached state; a locked state; an armed state; a standard sampling state; and an extended sampling state.

In Example 3, the subject matter of Example 2 includes, wherein the needle inlet guide tube comprises: at least one engagement ramp configured to engage with the needle actuator as the sampling device moves between the locked state, the armed state, the standard sampling state, and the extended sampling state; and at least one groove configured to engage with the needle actuator to maintain the sampling device in one or more of the operating states.

In Example 4, the subject matter of Example 3 includes, wherein the needle actuator comprises: a housing extending longitudinally along a central axis between a first end portion and a second end portion; a first button assembly extending at least partially outside of the housing such that the first button assembly is engageable from outside the housing; and a second button assembly extending at least partially outside of the housing such that the second button assembly is engageable from outside the housing.

In Example 5, the subject matter of Example 4 includes, wherein the first button assembly comprises a first engagement feature that is engageable with the needle inlet guide tube.

In Example 6, the subject matter of Example 5 includes, wherein the first button assembly comprises a spring that biases the first button assembly such that the first engagement feature at least partially extends into the working lumen.

In Example 7, the subject matter of Example 6 includes, wherein the needle inlet guide tube extends between a third end portion and a fourth end portion, wherein the at least one engagement ramp comprises a first engagement ramp adjacent to the third end portion of the needle guide tube and a second engagement ramp, and wherein the at least one groove comprises a first groove between the first engagement ramp and the second engagement ramp.

In Example 8, the subject matter of Example 7 includes, wherein the first engagement feature of the first button assembly engages with the first engagement ramp or the second engagement ramp and catches in the first groove to move the sampling device into the locked state such that the needle actuator cannot move relative to the needle inlet guide tube.

In Example 9, the subject matter of Example 8 includes, wherein the tip of the sampling needle is retracted five or more millimeters within the sampling device when the sampling device is in the locked state.

In Example 10, the subject matter of Examples 7-9 includes, wherein the at least one engagement ramp further include: a third engagement ramp; and a trough feature between the second engagement ramp and the third engagement ramp.

In Example 11, the subject matter of Example 10 includes, wherein engaging the first button assembly and sliding the needle actuator relative to the needle inlet guide tube such that the first engagement feature engages with the second engagement ramp or the third engagement ramp and catches in the trough feature puts the sampling device in the armed state such that no more buttons need to be engaged to put the sampling device into the standard sampling state.

In Example 12, the subject matter of Example 11 includes, wherein the tip of the sampling needle is retracted within the sampling device about three to five millimeters when the sampling device is in the armed state.

In Example 13, the subject matter of Examples 10-12 includes, wherein the at least one groove comprises a standard sampling groove adjacent to the third engagement ramp and extending toward the fourth end portion.

In Example 14, the subject matter of Example 13 includes, wherein sliding the needle actuator relative to the needle inlet guide tube such that the first engagement feature engages the third engagement ramp and catches in the standard sampling groove, puts the sampling device in the standard sampling state.

In Example 15, the subject matter of Example 14 includes, wherein in the standard sampling state, the first engagement feature engaging with the third engagement ramp and the third end portion of the needle inlet guide tube engaging with the second button assembly defines a first travel limit for the standard sampling state.

In Example 16, the subject matter of Example 15 includes, wherein the first travel limit allows the tip of the sampling needle to extend from the sampling device between zero and ten millimeters when the sampling device is in the standard sampling state.

In Example 17, the subject matter of Examples 15-16 includes, wherein engaging the second button assembly such that a second base of the second button assembly retracts outside of the working lumen and sliding the needle actuator relative to the needle inlet guide tube such that the third end portion of the needle inlet guide tube clears the second base moves the sampling device into the extended sampling state.

In Example 18, the subject matter of Example 17 includes, wherein in the extended sampling state the first engagement feature engaging with third engagement ramp and the third end portion engaging with the base of the sampling needle defines a second travel limit for the extended sampling state.

In Example 19, the subject matter of Example 18 includes, wherein the second travel limit allows the tip of the sampling needle to extend from the sampling device between zero and twenty millimeters when the sampling device is in the extended sampling state.

In Example 20, the subject matter of Examples 17-19 includes, wherein the second button assembly comprises a lock stop extending laterally outward from the second base and including a lock surface that is engageable to maintain the second button assembly in an engaged position.

In Example 21, the subject matter of Example 20 includes, wherein the sampling device comprises a locking mechanism cradled in the housing of the needle actuator and configured to maintain the second button assembly in the engaged position after the second button assembly is engaged by an operator of the sampling device.

In Example 22, the subject matter of Example 21 includes, wherein the locking mechanism comprises: a pivotable rod extending between a first end and a second end, the pivotable rod configured to rotate the locking mechanism in a clockwise direction and a counterclockwise direction; a spring base extending from the first end of the pivotable rod, the spring base including a locking spring configured to engage with the housing of the needle actuator to rotate the pivotable rod in the clockwise direction and position the locking mechanism into a locked position; and a lock catch extending from the second end of the pivotable rod and engageable with the lock stop of the second button assembly when the second button assembly is in the engaged position to prevent the locking mechanism from rotating in the clockwise direction, the lock catch further engageable with the lock surface when the second button assembly is in the engaged position to maintain the second button assembly in the engaged position.

In Example 23, the subject matter of Example 22 includes, wherein the locking mechanism comprises an unlock flag extending from the first end of the pivotable rod, the unlock flag is engageable to rotate the locking mechanism in the counterclockwise direction.

In Example 24, the subject matter of Example 23 includes, wherein the first button assembly comprises an extended sampling state exit tab that extends longitudinally from the first button assembly toward the first end of the housing, and wherein the extended sampling state exit tab is engageable with the unlock flag of the locking mechanism to rotate the locking mechanism in the counterclockwise direction to disengage the lock catch from the lock surface of the lock stop.

In Example 25, the subject matter of Examples 4-24 includes, wherein the sampling device further comprises a stylet configured to be installed within the housing of the needle actuator, the stylet configured to extend within a second working lumen of the sampling needle to prevent tissue from filling the second working lumen during insertion of the sampling needle.

Example 26 is a method of operating a sampling device for sampling target nodules in lungs of a patient, the method comprising: sliding a needle inlet guide tube within a working lumen of a needle actuator until a first engagement feature catches in a first notch, thereby placing the sampling device in a locked state where the needle actuator cannot translate relative to the needle inlet guide tube; compressing a first button assembly and moving the needle actuator relative to the needle inlet guide tube until the first engagement feature settles into a trough feature to put the sampling device in an armed state; and sliding the needle actuator relative to the needle inlet guide tube until the first engagement feature extends into a standard sampling groove to put the sampling device in a standard sampling state.

In Example 27, the subject matter of Example 26 includes, wherein a standard travel limit allows a tip of a sampling needle to extend from the sampling device between zero to ten millimeters when the sampling device is in the standard sampling state.

In Example 28, the subject matter of Examples 26-27 includes, engaging a second button assembly to retract a second base outside of the working lumen; and sliding the needle actuator relative to the needle inlet guide tube so that a first end portion of the needle inlet guide tube clears the second base of the second button assembly to enter an extended sampling state.

In Example 29, the subject matter of Example 28 includes, wherein an extended travel limit allows a tip of the sampling needle to extend from the sampling device zero to twenty millimeters when the sampling device is in the extended sampling state.

Example 30 is a sampling device insertable into a patient to obtain samples of target portions of a patient, the sampling device comprising: a medical instrument for obtaining a sample extending between a base and a tip; a medical instrument guide tube defining a working lumen; and an actuator configured to receive the base of the medical instrument to extend the medical instrument through the working lumen, the actuator engageable with the medical instrument guide tube to move the medical instrument through the sampling device; the sampling device including operating states, in each of the operating states the actuator moves relative to the medical instrument guide tube to move the tip of the medical instrument with respect to the sampling device.

Example 31 is an apparatus comprising means to implement of any of Examples 1-30.

Example 32 is a system to implement of any of Examples 1-30.

Example 32 is a method to implement of any of Examples 1-30.

The above-detailed description includes references to the accompanying drawings, which form a part of the detailed description. The drawings show, by way of illustration, specific examples that can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description, with each claim standing on its own as a separate example. The scope of the examples should be determined with reference to the appended claims, along with the full scope of equivalents to which such claims are entitled.

1. A sampling device insertable into a bronchoscope to obtain samples of target nodules in lungs of a patient, the sampling device comprising:
a sampling needle extending between a base and a tip;
a needle inlet guide tube removably couplable to the bronchoscope and defining a working lumen; and
a needle actuator configured to receive the base of the sampling needle to extend the sampling needle through the working lumen, the needle actuator engageable with the needle inlet guide tube to move the sampling needle through the sampling device;
the sampling device including operating states, in each of the operating states the needle actuator moves relative to the needle inlet guide tube to move the tip of the sampling needle with respect to the sampling device.

2. The sampling device of claim 1, wherein the operating states comprise:
a detached state;
a locked state;
an armed state;
a standard sampling state; and
an extended sampling state.

3. The sampling device of claim 2, wherein the needle inlet guide tube comprises:
at least one engagement ramp configured to engage with the needle actuator as the sampling device moves between the locked state, the armed state, the standard sampling state, and the extended sampling state; and
at least one groove configured to engage with the needle actuator to maintain the sampling device in one or more of the operating states; and
wherein the needle actuator comprises:
a housing extending longitudinally along a central axis between a first end portion and a second end portion;
a first button assembly extending at least partially outside of the housing such that the first button assembly is engageable from outside the housing; and
a second button assembly extending at least partially outside of the housing such that the second button assembly is engageable from outside the housing.

4. The sampling device of claim 3, wherein the first button assembly comprises a first engagement feature that is engageable with the needle inlet guide tube.

5. The sampling device of claim 4, wherein the first button assembly comprises a spring that biases the first button assembly such that the first engagement feature at least partially extends into the working lumen.

6. The sampling device of claim 5, wherein the needle inlet guide tube extends between a third end portion and a fourth end portion, wherein the at least one engagement ramp comprises a first engagement ramp adjacent to the third end portion of the needle inlet guide tube and a second engagement ramp, and wherein the at least one groove comprises a first groove between the first engagement ramp and the second engagement ramp.

7. The sampling device of claim 6, wherein the first engagement feature of the first button assembly engages with the first engagement ramp or the second engagement ramp and catches in the first groove to move the sampling device into the locked state such that the needle actuator cannot move relative to the needle inlet guide tube.

8. The sampling device of claim 7, wherein the tip of the sampling needle is retracted five or more millimeters within the sampling device when the sampling device is in the locked state.

9. The sampling device of claim 6, wherein the at least one engagement ramp further include:
a third engagement ramp; and
a trough feature between the second engagement ramp and the third engagement ramp.

10. The sampling device of claim 9, wherein engaging the first button assembly and sliding the needle actuator relative to the needle inlet guide tube such that the first engagement feature engages with the second engagement ramp or the third engagement ramp and catches in the trough feature puts the sampling device in the armed state such that no more buttons need to be engaged to put the sampling device into the standard sampling state.

11. The sampling device of claim 10, wherein the tip of the sampling needle is retracted within the sampling device about three to five millimeters when the sampling device is in the armed state.

12. The sampling device of claim 9, wherein the at least one groove comprises a standard sampling groove adjacent to the third engagement ramp and extending toward the fourth end portion, and wherein sliding the needle actuator relative to the needle inlet guide tube such that the first engagement feature engages the third engagement ramp and catches in the standard sampling groove, puts the sampling device in the standard sampling state.

13. The sampling device of claim 12, wherein in the standard sampling state, the first engagement feature engaging with the third engagement ramp and the third end portion of the needle inlet guide tube engaging with the second button assembly defines a first travel limit for the standard sampling state.

14. The sampling device of claim 13, wherein the first travel limit allows the tip of the sampling needle to extend from the sampling device between zero and ten millimeters when the sampling device is in the standard sampling state.

15. The sampling device of claim 13, wherein engaging the second button assembly such that a second base of the second button assembly retracts outside of the working lumen and sliding the needle actuator relative to the needle inlet guide tube such that the third end portion of the needle inlet guide tube clears the second base moves the sampling device into the extended sampling state.

16. The sampling device of claim 15, wherein in the extended sampling state the first engagement feature engaging with third engagement ramp and the third end portion engaging with the base of the sampling needle defines a second travel limit for the extended sampling state.

17. The sampling device of claim 16, wherein the second travel limit allows the tip of the sampling needle to extend from the sampling device between zero and twenty millimeters when the sampling device is in the extended sampling state.

18. The sampling device of claim 15, wherein the second button assembly comprises a lock stop extending laterally outward from the second base and including a lock surface that is engageable to maintain the second button assembly in an engaged position.

19. The sampling device of claim 18, wherein the sampling device comprises a locking mechanism cradled in the housing of the needle actuator and configured to maintain the second button assembly in the engaged position after the second button assembly is engaged by an operator of the sampling device.

20. The sampling device of claim 19, wherein the locking mechanism comprises:
a pivotable rod extending between a first end and a second end, the pivotable rod configured to rotate the locking mechanism in a clockwise direction and a counterclockwise direction;
a spring base extending from the first end of the pivotable rod, the spring base including a locking spring configured to engage with the housing of the needle actuator to rotate the pivotable rod in the clockwise direction and position the locking mechanism into a locked position; and
a lock catch extending from the second end of the pivotable rod and engageable with the lock stop of the second button assembly when the second button assembly is in the engaged position to prevent the locking mechanism from rotating in the clockwise direction, the lock catch further engageable with the lock surface when the second button assembly is in the engaged position to maintain the second button assembly in the engaged position.

21. The sampling device of claim 20, wherein the locking mechanism comprises an unlock flag extending from the first end of the pivotable rod, the unlock flag is engageable to rotate the locking mechanism in the counterclockwise direction.

22. The sampling device of claim 21, wherein the first button assembly comprises an extended sampling state exit tab that extends longitudinally from the first button assembly toward the first end of the housing, and wherein the extended sampling state exit tab is engageable with the unlock flag of the locking mechanism to rotate the locking mechanism in the counterclockwise direction to disengage the lock catch from the lock surface of the lock stop.

23. A method of operating a sampling device for sampling target nodules in lungs of a patient, the method comprising:
sliding a needle inlet guide tube within a working lumen of a needle actuator until a first engagement feature catches in a first notch, thereby placing the sampling device in a locked state where the needle actuator cannot translate relative to the needle inlet guide tube;
compressing a first button assembly and moving the needle actuator relative to the needle inlet guide tube until the first engagement feature settles into a trough feature to put the sampling device in an armed state; and
sliding the needle actuator relative to the needle inlet guide tube until the first engagement feature extends into a standard sampling groove to put the sampling device in a standard sampling state.

24. The method of claim 23, wherein a standard travel limit allows a tip of a sampling needle to extend from the sampling device between zero to ten millimeters when the sampling device is in the standard sampling state.

25. The method of claim 24, further comprising:
engaging a second button assembly to retract a second base outside of the working lumen; and
sliding the needle actuator relative to the needle inlet guide tube so that a first end portion of the needle inlet guide tube clears the second base of the second button assembly to enter an extended sampling state;
wherein an extended travel limit allows a tip of the sampling needle to extend from the sampling device zero to twenty millimeters when the sampling device is in the extended sampling state.
